(19) United States
(12) Patent Application Publication
(10) Pub. No.: US 2004/0049227 A1
(43) Pub. Date: Mar. 11, 2004

(54) Helical fastener and applicator for surgical procedures

(76) Inventor: James E. Jervis, Atherton, CA (US)

Correspondence Address: Chief Patent Counsel, US Surgical, a Division of Tyco Healthcar Group LP, 150 Glover Avenue, Norwalk, CT, 06856, US

(21) Appl. No.: 10/640,179

(22) Filed: Aug. 13, 2003

Related U.S. Application Data
(63) Continuation of application No. 10/037,200, filed on Oct. 25, 2001, now abandoned.
Application No. 10/037,200 is a continuation of application No. 09/535,941, filed on Mar. 24, 2000, now abandoned.
Application No. 09/535,941 is a continuation of application No. 09/195,312, filed on Nov. 18, 1998, now abandoned.

(51) Int. Cl. A61B 17/10

(52) U.S. Cl. 606/213; 606/143

(57) ABSTRACT

A device and method for deploying helical fasteners which may be used to attach a prosthesis to tissue, e.g., to attach a mesh patch in a hernia repair procedure. A helical coil fastener which is inhibited from undesirable migration after installation is also provided.

### FIELD OF THE INVENTION

The present invention relates to surgical fasteners. In particular, the invention relates to helical fasteners and a means for containing and applying a plurality of them in the course of laparoscopic surgery.

### BACKGROUND OF THE INVENTION

The time consuming processing of traditional suturing necessitated the development of surgical fasteners. By shortening the time of the suturing process, surgical fasteners reduce blood loss and trauma to the patient. For open surgery, there has been a substantial amount of activity in developing staples and staple applicators to be used to secure tissue elements, e.g., in wound closure. Staples have also found use in attaching prostheses, e.g., the mesh patches used to repair hernias.

The use of staples has now become widespread, but with the advent of minimally invasive, or laparoscopic, surgery, the amount of room available for fastening devices to operate in has decreased substantially. In addition, in some instances, e.g., laparoscopic hernia repair, the surgeon has access to only one side of the tissue to which the mesh patch is to be secured.

Furthermore, stapling of a mesh patch in a hernia repair procedure generally requires the exertion of axial force on the staple and the creation of a counter-force (such as that provided by an anvil when a stapler is used in open surgery) can be difficult. Thus, a need arose for a fastening means and applicator system which would not be subject to these difficulties.

One answer to these problems was provided by the use of helical coil fasteners, such as those described in Gianturco U.S. Pat. No. 5,258,000 and Bolduc U.S. Pat. Nos. 5,582,616; 5,810,882; 5,824,008; the disclosures of which are incorporated herein by reference in their entireties, which were developed as an alternative to staple fasteners. Some suture devices have also made use of helically coiled components, such as those disclosed in Yoon U.S. Pat. No. 5,053,047 and Buzerak U.S. Pat. No. 5,356,424, the disclosures of which are incorporated herein in their entireties. Since a helical fastener is driven through the items to be attached, e.g., a mesh patch and underlying tissue, by the rotational driving force of the helical coil, the need for axial pressure is greatly reduced. The Bolduc embodiment of a helical coil fastener device includes a helical coil and an applicator. The helical coil has a distal end adapted to penetrate tissue and a proximal end capable of receiving longitudinal and rotational force from the applicator. The applicator can comprise a stationary hollow tube adapted to contain one or more fasteners. Additionally, there may be a movable rod within and sharing an axis with the hollow tube. The rod may have a groove running longitudinally along its length or be “D” shaped, whereby it is adapted to engage the fastener. The rod may further be adapted to be able to apply longitudinal and rotational force to the fastener. Helical coil fasteners may be applied to tissue accessible from one side and, also, have a large retentive surface area for securing a prosthesis, e.g., a mesh, to the tissue.

The manner in which the helical coil engages a rotator designed to impart rotational motion to the coil is important for ease of use. The present invention, unlike previous helical coil fastener systems, employs a groove in the rotator which is engaged by a protuberance extending from the helical coil. Such an arrangement has the advantages of simplicity and ease of use. Additionally and separately, the present invention also contemplates providing a helical coil fastener which is provided with means designed to prevent migration of the coil out of its desired location after it has been installed.

### SUMMARY OF THE INVENTION

The present invention is directed to an improved fastener and applicator for inserting the fastener into tissue.

The preferred embodiment of the fastener comprises a short helical coil. At the distal end of the wire or similar material forming the coil is a point or other configuration suitable for passing through mesh and piercing tissue. At the proximal end of the coil is a protuberance extending outside the circumference of the helix, radially or obliquely. The protuberance must be extensive enough to engage a first coil engaging means such as a slot or keyway in or on the interior surface of the distal end of an applicator tube. Preferably, the protuberance does not extend beyond the exterior surface of the applicator tube. The fastener may be made of any material suitable for medical implantation, including but not limited to metal, plastic or absorbent materials. Titanium is a preferred material.

An alternate embodiment of the fastener provides a loop at the proximal end to prevent the fastener from continued migration through the tissue and/or to prevent disattachment of a mesh or other prosthetic element attached to the tissue by the fastener. The loop interrupts the continuous form of the helix, thereby providing greater resistance to undesired movement.

When the distal end of the fastener is held against or in proximity to a mesh patch or other entity which is to be attached to tissue into which the fastener is to be placed, and the fastener is turned about its longitudinal axis, it will advance helically and pass through the mesh or other entity and into the tissue thereby attaching such entity to the tissue.

The preferred embodiment of the applicator comprises a delivery tube that is adapted to contain a plurality of fasteners, but also contemplates a device having a delivery means adapted to hold only one fastener at a time. The applicator tube also functions as a driving means to rotate the fastener contained therein. The applicator tube is slotted or grooved along its longitudinal axis. This slot or groove engages the protuberance on the proximal portion of the fastener, allowing the tube to exert rotational force on the fastener. The length of the tube must be suitable to reach the operative site from outside the patient. The inside diameter of the tube should be large enough to allow translation of the fasteners contained therein while at the same time being small enough to maintain driving contact with the protuberance on the fastener.

The preferred embodiment further comprises a stationary stabilizer rod. The rod is contained within the tube. The rod is sized to slide within the helical coil of the fasteners. At or near the distal tip of the rod is a coil engaging means which engages the fastener. This means may be a radially outwardly extending pin, a thread form matching the helical coil of the fastener, a plurality of radially outwardly extending pins positioned to match the helical coil of the fastener or any other form that functions to engage the fastener. Once the protuberance on the fastener is in engagement with the slot in the tube and the coil of the fastener is in engagement with the coil engaging means, the tube is rotated, thereby rotating the fasteners, and the fasteners are thus advanced in the manner of a screw. If a multiple fastener device is desired, the fasteners may be stacked successively within a tube of desired length and in such a device the rod and coil engaging means will preferably be lengthened to engage all fasteners such that all are distally advanced together upon rotation of the applicator tube.

In an alternate embodiment, a loading spring may operate in the annulus between the tube and rod. The loading spring functions to bias the fasteners toward the distal tip of the applicator tube. As a result, upon rotation of the tube, the fasteners are successively urged into engagement with the coil engaging means at or near the distal portion of the rod. The loading spring must be of adequate length and sufficient force to cause all fasteners, in turn, to engage the coil engaging means on the rod. Preferably, the loading spring may rotate with the tube. In an alternate embodiment, the loading spring may be stationary with respect to the tube. Advantageously, a means to provide a non-tangling interface such as a washer is provided at the distal end of the loading spring and is in contact with the most proximal fastener and may also optionally provide a visual indication when all fasteners have been ejected.

The applicator further comprises a handle at the proximal end of the applicator. A suitable handle must fix the stabilizer rod and must rotate the applicator tube. The handle may, advantageously, rotate a measured amount to eject one fastener at a time. Preferably, the handle further comprises a clutch mechanism which prevents counter-rotation of the tube.

Although helical coil fasteners are preferred, other shapes which may be used according to the present invention include any suitable circular or cylindrical or conical shape in which generally circular loops are used.

Other objects and advantages of the present inventions will become apparent from the detailed description of the preferred embodiments which follow.

### BRIEF DESCRIPTION OF THE DRAWINGS

FIG. 1 shows a slide view of a rotatable hollow tube according to the invention, with a slot running longitudinally along the distal portion.

FIG. 2 shows a side view of a stationary stabilizer rod according to the invention, with a coil engaging means near the distal tip and a loading spring and washer near the proximal end.

FIG. 3 shows a side view of a fastener according to the invention.

FIG. 4 shows an end view of a fastener according to the invention.

FIG. 5 shows an end view of a fastener engaged in a rotatable hollow tube according to the invention. A protuberance extending from the blunt end of the spring mates with a longitudinal slot in the tube.

FIG. 6 shows a schematic partial cross-sectional view of the interrelationship between a fastener, a rotatable hollow tube, and a stationary stabilizer rod.

FIG. 7 shows an embodiment of the applicator device used to deploy the fastener.

FIG. 8 shows an alternate embodiment of a rotatable hollow tube having a slot or keyway that runs longitudinally along the distal portion.

FIG. 9 shows an alternate embodiment of a stationary stabilizer rod with a thread form matching the fastener helix located near the distal tip.

FIGS. 10a and 10b show an alternate embodiment of a fastener with a loop near the blunt end.

FIG. 11 shows an end view of an alternate embodiment of a fastener according to the invention.

### DETAILED DESCRIPTION OF THE PREFERRED EMBODIMENTS

As is shown in the drawings, which are offered for purposes of illustration and not be way of limitation, the invention is embodied by a helical fastener and an applicator therefor. One embodiment of the applicator of the present invention, as seen in FIGS. 1-5, comprises a rotatable hollow tube 10 that drives a fastener 30 into tissue. The tube 10 must be of a length suitable to reach the operative site from outside the patient. The fastener 30 may be further guided by a stationary stabilizer rod 20. It is preferred that the rotatable hollow tube 10 and the stationary stabilizer rod 20 are of such dimensions that the fasteners 30 can be placed between and in contact with both the tube 10 and the rod 20 and also slide without excess friction along the rod 20 and within the tube as shown in FIG. 6.

In a preferred embodiment of the invention (See FIG. 1), the rotatable hollow tube 10 has a longitudinal slot 12 along a distal portion 14 thereof. The slot 12 may of any length, but preferably is of a length equal to or greater than the length of the fasteners in tube 10. It is also desirable that slot 12 be evenly divisible by one fastener length.

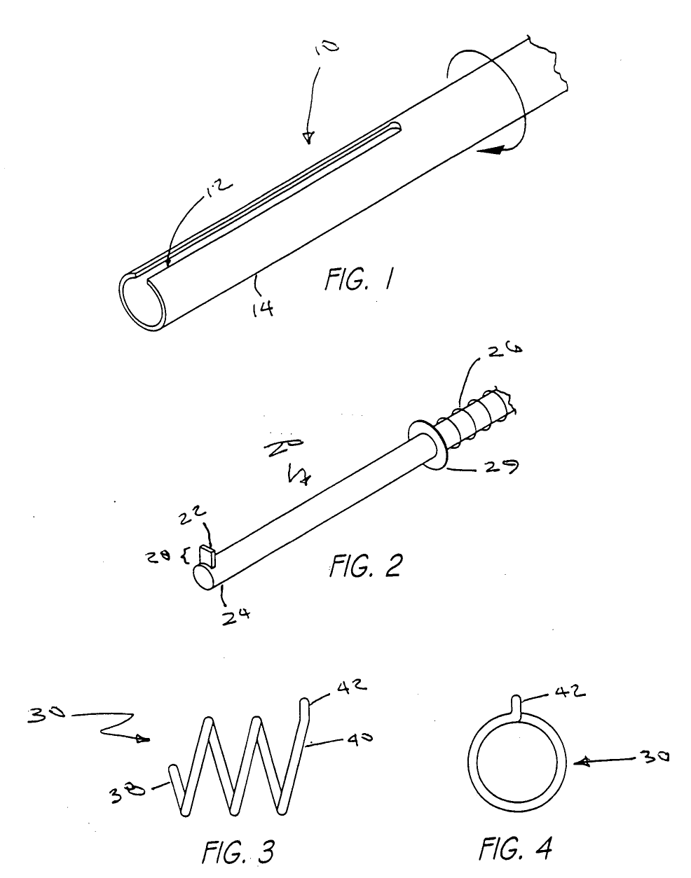

In one preferred embodiment of the invention (See FIG. 2), the stationary stabilizer rod 20 has a second coil engaging means such as pin 22 at the distal portion 24 of the rod. The pin 22 may be of any height 28, but is preferably of such a height that the pin 22 is flush with the outer portion of the rotatable hollow tube 10. The pin 22 is shown as having a generally square shape, but may be of any shape capable of engaging a fastener 30.

As seen in FIGS. 2 and 6, a loading spring 26 may operate at the proximal portion of the rod 20. The loading spring 26 acts to bias the fasteners 30 toward the pin 22. Advantageously, the loading spring 26 may rotate with the tube 10. However, the loading spring 26 may be stationary. The loading spring 26 should be of sufficient length and adequate force to urge all fasteners 30 in turn into engagement with second coil engaging means 22. Preferably, a washer 20 may be located at the distal tip of the loading spring 26. The washer 29 functions to provide a non-tangling interface with the most proximal of the fasteners 30 and, optionally, to provide visual indication when all fasteners 30 have been ejected. Additionally, in place of the washer 29, any other suitable means that may provide a non-tangling interface or visual indication may be used.

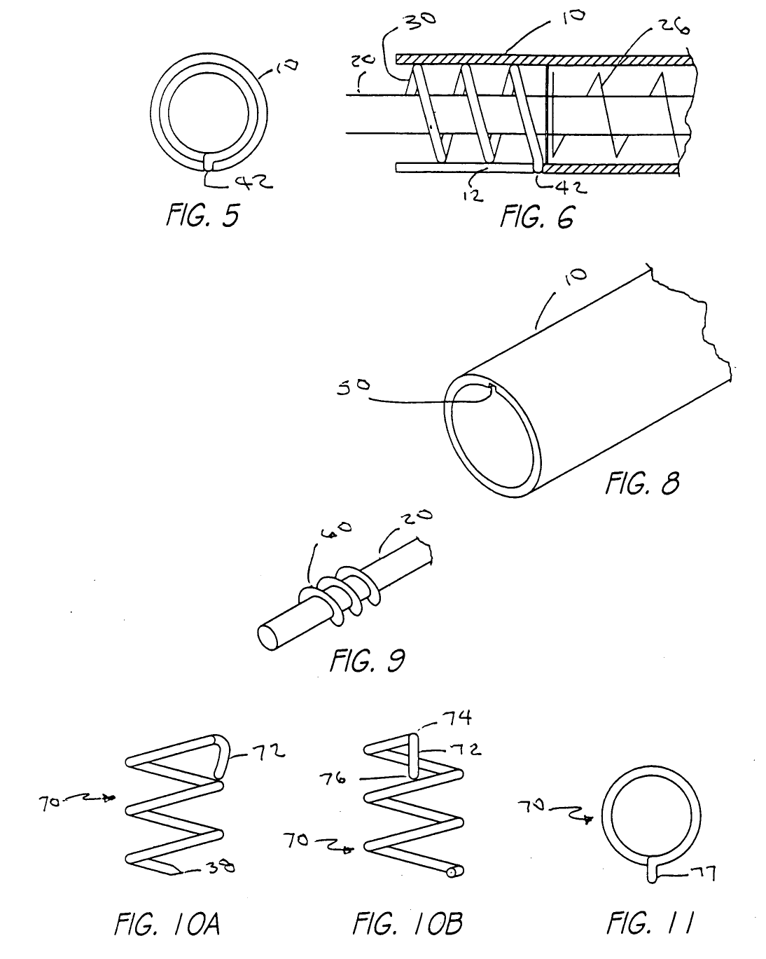

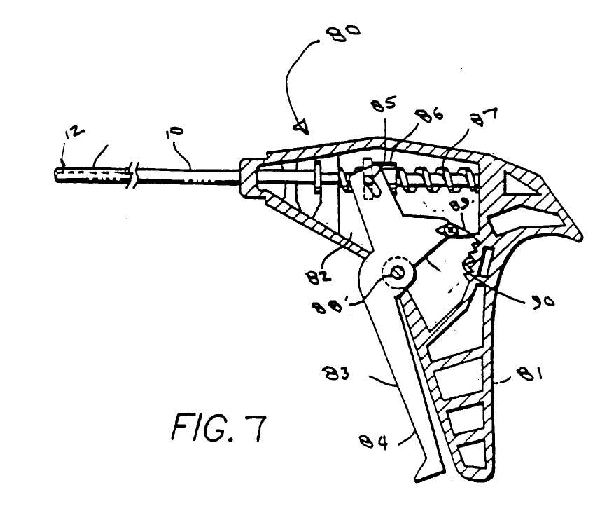

In a preferred embodiment of the invention (See FIGS. 3 and 4), the fastener 30 has a helical shape. The length, pitch, and diameter of the coil may be varied depending upon the application. Additionally, the fastener 30 may be of any suitable circular, cylindrical or conical shape in which generally circular loops are used. Moreover, the fastener 30 has a piercing end 38 that may be of varying form or sharpness, depending on the application. The proximal end 40 of the fastener 30 should have a protuberance 42 extending beyond the diameter 36 of the fastener. The protuberance 42 may extend any length outside the diameter 36 of the fastener 30, but preferably extends a length less than or equal to the thickness of the rotatable hollow tube 10 (See FIGS. 5 and 6).

As shown in FIG. 7, applicator 80 has a handle 81 and an actuator 82. The applicator is equipped with a tube member such as tube 10 having a slot such as slot 12. In operation, the actuator 82 functions to rotate tube 12 which drives the helically coiled fastener out of the applicator and into the tissue of the patient. The applicator is provided with lever 83. As shown in FIG. 7, the lower end 84 of the lever 83 is a trigger-like member suitable for manual manipulation. The other, or upper, end 85 of lever 83 engages driver 86. Lead screw 87 which has a helical thread thereon is rotatably mounted in the applicator and engages driver 62. The longitudinal axis of tube 10 is coaxial with the longitudinal axis of screw 87.

When the trigger end 84 of the lever 83 is squeezed, the driver is moved along screw 87 causing it to rotate. This, in turn causes, tube 12 to rotate thereby causing the helical coil fasteners in tube 12 to move distally through the tube. Preferably, the length of travel of the coil fasteners resulting from one squeeze of the trigger will be just enough to force one coil fastener out of tube 12 and will then leave the next fastener at the distal end of tube 12 ready to be discharged on the next trigger squeeze.

When trigger portion 84 is squeezed, lever 83 pivots on its center of rotation 88. Lever 83 can be provided with a spring loaded pawl 89 adapted to engage teeth 90 such that one full pull of the trigger is required before the trigger can be returned to its undepressed position. This assures that each pull of the trigger will fully discharge one fastener.

In one alternate embodiment of the invention shown in FIG. 8, the rotatable hollow tube 10 has a longitudinal groove or keyway 50 along the inner surface of tube 10 instead of a slot in the tube. The groove 50 acts to engage the protuberance 42 of the fastener 30, thereby applying rotational force to the fastener 30.

In another alternate embodiment of the invention, as seen in FIG. 9, the stationary stabilizer rod 20 has a thread form 60 that mates with the coil of the fastener 30. Specifically, the thread form 60 engages the fastener helix, thereby cooperating with rotation of tube 10 to advance the fastener 30 into tissue in a screw-like manner upon rotation of tube 10. Optionally, a plurality of pins may be positioned to form a thread that mates with the fastener helix. If the length of the thread is at least as long as the stack of fasteners carried in the tube 10, the load spring may be eliminated.

In still another alternate embodiment of the invention, as seen in FIGS. 10a and 10b, a fastener 70 may have a loop portion 72 near the blunt end. Preferably, the loop portion 72 has a connected end 74 and a free end 76. Optionally, both ends may be connected (not shown). In the embodiment having only one connected end, the loop portion 72 may extend beyond the diameter of the helical body of the fastener 70 at the connected end 74. The loop portion 72 may extend any length beyond the diameter of the fastener 70, but preferably extends a distance less than or equal to the thickness of the rotatable hollow tube 10.

The alternate fastener embodiment shown in FIG. 1I has a trailing tail 79 which extends outside tube 10 and is driven by the offset portion of tail 79 which engages slot 12.

Certain embodiments have been described herein, and are illustrated in the drawings. However, it will be apparent to those skilled in the art that modifications can be made to the embodiments without departing from the inventive concepts described. Accordingly, the invention is not be to restricted except by the claims which follow.

What is claimed is:

1. An applicator for inserting a fastener into tissue comprising:
a rotatable hollow outer tube having a proximal portion and a distal portion; and
a stationary stabilizer rod contained substantially within said tube;
a fastener mounted on said rod;
said tube having a longitudinally extending coil engaging means along a distal portion thereof, said tube further being adapted to contain a fastener in engaging relationship with said slot;
said rod having a member adapted to engage said fastener in the distal region of said rod.

2. The applicator of claim 1 wherein said coil engaging means is a slot and wherein said fastener comprises at least one helical coil having a radially outwardly extending protuberance is provided in said tube and wherein said protuberance engages with said slot.

3. The applicator of claim 2 wherein said member on the distal region of said rod is a coil engaging member.

4. The applicator of claim 3 wherein said coil engaging member is a pin which extends radially outwardly from said rod.

5. The applicator of claim 3 wherein said coil engaging member comprises screw threads on said rod.

6. An applicator for inserting a fastener into tissue comprising:
a hollow tube having an interior and exterior surface;
a substantially solid rod having a distal region; and
a helically coiled fastener mounted on said rod;
said tube being rotatable about the longitudinal axis thereof, and further having a longitudinal groove extending along said interior surface;
said rod being substantially enclosed by said tube, and having a member adapted to engage said fastener at its distal region; and,
said fastener having a protuberance in engagement with said groove.

7. The applicator of claim 6 wherein said member on the distal region of said rod is a coil engaging member.

8. The applicator of claim 7 wherein said coil engaging member is a pin which extends radially outwardly from said rod.

9. The applicator of claim 7 wherein said coil engaging member comprises screw threads on said rod.

10. A method of inserting a fastener into tissue comprising the steps of:
providing a fastener with a piercing end and a blunt end;
engaging the fastener with a means which will cause the fastener to advance when it is rotated,
providing a tube containing at least one fastener which tube is rotatable about the longitudinal axis thereof;
putting the fastener in engaging relationship with the tube;
rotating the tube, whereby the fastener is translated into the tissue in a screw like manner.

11. A fastener to stabilize tissue comprising:
a helical body having at least one coil, said body having a proximal portion and a distal portion;
said distal portion being suitable for piercing tissue;
said proximal end having a loop portion extending from a first point on said coil to a second point on said coil, said proximal end further having a protuberance extending outside an area defined by the perimeter of said helical body.
